(19) United States
(12) Patent Application Publication
(10) Pub. No.: US 2025/0229246 A1
(43) Pub. Date: Jul. 17, 2025

(54) REDUCING MAINTENANCE AND INCREASING ENERGY SAVINGS IN THE PRODUCTION OF A CHEMICAL REACTION PRODUCT INVOLVING HEAT RECOVERY

(71) Applicant: BASF SE, Ludwigshafen am Rhein (DE)

(72) Inventors: Andreas KELLER, Ludwigshafen am Rhein (DE); Rupert WAGNER, Ludwigshafen am Rhein (DE); Volker Gerhard DINGES, Shanghai (CN); Martin KAMASZ, Ludwigshafen am Rhein (DE)

(21) Appl. No.: 18/855,658

(22) PCT Filed: Apr. 11, 2023
(86) PCT No.: PCT/EP2023/059446

§ 371 Date: Oct. 10, 2024

(30) Foreign Application Priority Data
Apr. 12, 2022 (WO) PCT/CN2022/086290

(51) Int. Cl. B01J 19/00; C07C 29/38

(52) U.S. Cl. CPC B01J 19/0026; C07C 29/38; B01J 2219/00092; B01J 2219/00094; B01J 2219/00247

(57) ABSTRACT

A method for prolonging operation intervals between maintenance disruptions in the production of a chemical reaction product, comprising directing at least one reactant stream into a reactor; reacting the reactant(s) in the reactor at elevated temperature and pressure, whereby the chemical reaction product is obtained; withdrawing a stream of a hot chemical reaction product from the reactor; and heat-exchanging the stream of hot chemical reaction product with at least one of the reactant streams; wherein heat-exchanging is performed in at least two shell-and-tube heat exchangers; each of the heat exchangers comprising a plurality of tubes and a shell-side heat exchange passage; wherein the hot chemical reaction product is directed through the tubes of the heat exchangers; and the reactant is guided through the shell-side passage, and at least two of the heat exchangers are connected in series with regard to both the shell-side flow and the tube-side flow. By using two or more heat exchangers, the impact of fouling in individual tubes on the overall heat exchange capacity is reduced in comparison to arrangements where only a single heat exchanger is used.

In a chemical process, various waste heat sources may be available, e.g., from the cooling of intermediate product streams or flue gas of any combustion process. Heat recovery during the production of a chemical reaction product is highly desirable. Heat available at a temperature of at least 400 to 500° C. is usually recovered by producing hot steam, which can be used in the process itself, or expanded in a turbine to produce energy. Heat available at a lower temperature is generally not suitable to produce energy and can be recovered, e.g., by pre-heating of one or more process stream(s). The pre-heating generally occurs by indirect heat exchange of a hot chemical reaction product stream with a reactant stream, thereby transferring heat to the reactant stream.

The temperature of the reactant stream preheated by indirect heat exchange with the hot chemical reaction product may be further adjusted, typically further increased in a heater before introducing the reactant stream into the reactor. Any means for adjusting the temperature may be applied. Such means include, without limitation, electrical heating, direct firing, and/or exchanging heat against another medium such as steam or hot oil.

One type of heat exchanger which is commonly used in connection with commercial processes is the shell-and-tube heat exchanger. In exchangers of this type, one fluid flows through the inside of the tubes, while the other fluid is forced through the shell and over the outside of the tubes. Typically, baffles are installed to support the tubes and to force the fluid across the tube bundle in a serpentine fashion.

One of the most problematic aspects associated with the use of heat exchangers is the tendency towards fouling. Fouling refers to the various deposits and coatings which form on the surfaces of heat exchangers as a result of process fluid flow and heat transfer. There are various types of fouling including corrosion, mineral deposits, polymerization, crystallization, coking, sedimentation and biological. In the case of corrosion, the surfaces of the heat exchanger can become corroded as a result of the interaction between the process fluids and the materials used in the construction of the heat exchanger. The situation is made even worse due to the fact that various fouling types can interact with each other to cause even more fouling. Fouling results in additional resistance with respect to the heat transfer, and thus decreased performance with respect to heat transfer. Fouling also causes an increased pressure drop in connection with the fluid flowing on the inside of the exchanger.

One particular area prone to fouling in conventional shell-and-tube heat exchangers is the tube area near the tube sheet near the inlet where the tube-side fluid leaves the individual tubes. Excessive fouling in this area can cause clogging of individual tubes and fluid stagnation along the entire length of these tubes. The fluid stagnation generally leads to reduced heat-transfer performance.

As a further consequence of the decreased heat transfer performance caused by fouling, the energy required in a heater to adjust the temperature of the pre-heated reactant stream to the desired reaction temperature increases. Consequently, more additional external heat becomes necessary which is detrimental in terms of energy demand and process economy, and often has a negative impact on the carbon dioxide footprint of the product.

A preferred but not exclusive application of the invention is extended heat recovery, more particularly the prolongation of operation intervals between maintenance disruptions in a process and plant for the synthesis of isoprenol. Isoprenol, or 3-methyl-3-buten-1-ol, is an important intermediate for pharmaceuticals and aroma compounds, with a yearly global production of several thousand tons. Isoprenol is commercially synthesized by reacting formaldehyde with isobutylene. High temperatures are required to obtain a high isoprenol yield in uncatalyzed reactions of formaldehyde with isobutylene. Effective removal of the heat is critical for the product quality and process safety. The heat removed from the isoprenol is used for raising the temperature of isobutylene before it enters the reactor.

The problem underlying the present invention can be seen in devising a process and plant for maintaining adequate heat-transfer performance of shell-and-tube heat exchangers used for heat recovery in a chemical process even in the case where individual tubes of the heat exchanger fail due to fouling. The improved process should enable a prolongation of operation intervals between maintenance disruptions, reduce energy demand for adjusting the temperature of the pre-heated reactant stream to the reaction temperature and thus increase process economy.

The problem is solved by a method for extended heat recovery during the production of a chemical reaction product, more particularly prolonging operation intervals between maintenance disruptions in such a method, comprising
directing at least one reactant stream into a reactor;
reacting the reactant(s) in the reactor at elevated temperature and pressure, whereby the chemical reaction product is obtained;
withdrawing a stream of a hot chemical reaction product from the reactor; and
heat-exchanging the stream of hot chemical reaction product with at least one of the reactant streams;
wherein heat-exchanging is performed in at least two shell-and-tube heat exchangers, preferably in counter-current mode; each of the heat exchangers comprising a plurality of tubes and a shell-side heat exchange passage;
wherein the hot chemical reaction product is directed through the tubes of the heat exchangers; and the reactant is guided through the shell-side passage, and at least two of the heat exchangers are connected in series with regard to both the shell-side flow and the tube-side flow.

The term “maintenance disruptions” is intended to mean a shutdown of the process that becomes necessary at recurring intervals in order to clear the tubes of the heat exchanger that have been clogged by fouling. An indicator of a necessity of a maintenance disruption is typically when the reactants leaving the last heat exchanger are insufficiently pre-heated and that even a subsequent heater is hardly able to put in additional external heat into the reactant to bring the reactant to the required temperature before it enters the reactor. One aspect of the invention is that the pre-heating of the at least one reactant can be maintained for a longer time at levels high enough so that the desired temperature of the reactant(s) can easily be reached before the reactant(s) enter the reactor.

According to the invention, heat-exchanging is performed in at least two shell-and-tube heat exchangers, which are connected in series with regard to both the shell-side flow and the tube-side flow. Preferably, at least the first heat exchanger, more preferably each of the heat exchangers is operated in counter-current mode. The at least one reactant stream is directed through the shell-side passage of the first heat exchanger and subsequently through the shell-side passage of the second and all further heat exchangers; and the hot chemical reaction product is directed to the tubes of the heat exchangers in reverse order, relative to the direction of the at least one reactant stream.

By using two or more heat exchangers, the impact of fouling in individual tubes on the overall heat exchange capacity is reduced in comparison to arrangements where only a single heat exchanger is used. As a consequence, the heat transfer rates are maintained at a desired level for longer periods, hence prolonging operation intervals between maintenance disruptions, and the pre-heating of the at least one reactant stream requires less additional external heat compared to a plant with a single heat exchanger in an advanced state of fouling.

It is envisaged that process interruptions due to shutdown and maintenance will be required on a less frequent basis.

The physical state of the hot chemical reaction product is not particularly limited as long as it is a fluid stream. The hot chemical reaction product may be in a gaseous state, a super critical state or a liquid state. In one embodiment, the stream of the hot chemical reaction product is a liquid stream.

The invention addresses issues that may occur during indirect heat exchange in a shell-and-tube heat exchanger when the hot chemical reaction product is prone to fouling.

A chemical reaction product is considered “prone to fouling” if in a fouling test it will form deposits on the tube walls when it is circulated through the tubes of a single shell-and-tube heat exchanger against a coolant, for example a reactant stream in need of pre-heating, directed through the shell-side of the heat exchanger, eventually resulting in individual tubes to become clogged. As a result, the heat exchange efficacy is reduced, and the outlet temperature of the coolant is decreased. In the fouling test, heat exchanging reduces the temperature of the stream of hot chemical reaction product by 150° C. The single heat exchanger may be a heat exchanger having 300 tubes with an outer diameter of 14 mm, an inner diameter of 10 mm and a length of 10 m, resulting in an (outer) heat exchange surface of 130 m². A means for determining whether the single heat exchanger becomes clogged during operation may be to monitor the heat transfer coefficient k of the single heat exchanger over time during heat exchange at constant conditions. The heat transfer coefficient k may be determined as follows:

$$k = \dot{Q} / ( A \times Δ {T}_{log} )$$

with
\dot{Q}=Heat flow [W]
A=heat exchange surface
ΔT_{log}=logarithmic temperature difference between hot and cold stream

The chemical reaction product is considered “prone to fouling” if the heat transfer coefficient k of the single heat exchanger decreases by more than 15%, preferably by more than 30% within a time period of 180 days, preferably 150 days, more preferably 120 days and even more preferably 100 days.

Often, phase change of the hot chemical reaction product upon cooling is associated with a tendency to fouling. Hence, in embodiments, the hot chemical reaction product undergoes an at least partial phase change during the heat exchange. For example, the hot chemical reaction product may undergo a transition from supercritical to liquid, from gas to liquid or from liquid to suspension. Transition from liquid to suspension may occur if a solubility limit of an ingredient is decreased upon cooling resulting in precipitation of said ingredient.

Often, cooling the hot chemical reaction product by a large temperature margin is associated with a tendency to fouling. Hence, in embodiments, heat exchanging may reduce the temperature of the stream of hot chemical reaction product by at least 100° C., preferably in the range of from 120 to 180° C., more preferably in the range of from 130 to 160° C. In this scenario, the stream of hot chemical reaction product has a temperature difference which may be calculated from the tube-side inlet temperature at the last heat exchanger, e.g. of the second heat exchanger if two heat exchangers are connected in series, and the tube-side outlet temperature at the first heat exchanger.

By using two or more heat exchangers, the impact of gradual deterioration of the heat transfer of the first heat exchanger over time caused by fouling in individual tubes in the first heat exchanger, relative to the direction of the at least one reactant stream, on the overall heat exchange capacity is reduced in comparison to arrangements where only a single heat exchanger is used.

In an embodiment, the reduction of the overall heat transfer coefficient k of all heat exchangers connected in series is less than the reduction of the heat transfer coefficient k of the first heat exchanger, preferably by at least 10 percentage points. For example, if the heat transfer coefficient k of the first heat exchanger is reduced by 35%, the overall heat transfer coefficient k of all heat exchangers connected in series should be reduced by a value that is at least 10 percentage points lower than 35%, i.e. by less than 25%. The “first” heat exchanger is the first heat exchanger seen in the direction of the at least one reactant stream.

The hot chemical reaction product flows on the inside of the tubes, while the reactant is forced through the shell and over the outside of the tubes. The hot chemical reaction product may be directed through the tubes in either upwards direction or downwards direction; preferably in downwards direction. Typically, baffles are placed to support the tubes and to force the reactant across the tube bundle in a serpentine fashion. In its travel through the shell-side passage, the reactant is generally caused to follow a tortuous path, the major proportion of which is horizontal, while the overall travel direction of the reactant in the shell-side passage is vertical, counter-current to the direction of flow of the hot chemical reaction product inside of the tubes.

According to the invention, both the flow path of the stream of the hot chemical reaction product and the reactant stream flow path comprise at least two heat exchangers connected in series with regard to both the shell-side flow and the tube-side flow. Two or more heat exchangers placed in parallel may be connected in series with at least one heat exchanger.

This encompasses an embodiment wherein heat-exchanging is performed in three or more heat exchangers, wherein a first heat exchanger is connected in series with two or more second heat exchangers that are arranged in parallel to each other. In this respect, “first” heat exchangers and “second” heat exchangers are viewed in the flow direction of the shell-side flow.

In a preferred embodiment, heat-exchanging is performed in 2 to 6 heat exchangers connected in series, preferably in 2 to 5 heat exchangers, more preferably in 2 to 4 heat exchangers, most preferably in 2 to 3 heat exchangers. In view of the fact that any additional heat exchanger in excess of two entails higher investment and maintenance costs, and the benefit achievable by use of more than two heat-exchangers does not increase to the same extent, 2 heat exchangers are generally preferred.

The required total heat exchange surface area of all shell-and-tube heat exchanger connected in series depends on the specific heat exchange task. The heat exchange surface area of a heat exchanger corresponds to the sum of tube wall surfaces in contact with the shell-side flow. The ratio of the heat exchange surface area of each individual heat exchanger relative to the heat exchange surface area of each of the other individual heat exchangers is preferably in the range of 1:10 to 10:1, preferably 1:4 to 4:1, more preferably 1:2 to 2:1. For example, in the case of two shell-and-tube heat exchanger being connected in series, the ratio of their heat exchange surface area is preferably in the range of 1:10 to 10:1, more preferably 1:4 to 4:1, most preferably 1:2 to 2:1.

The heat exchangers typically each comprise 50 to 5000 tubes, more preferably 400 to 3000 tubes, most preferably 800 to 2000 tubes.

The tubes preferably have an inner diameter in the range of 6 to 25 mm, more preferably 8 to 16 mm, most preferably 8 to 12 mm.

Preferably, the tubes have a length of 2 to 30 m, for example 2 to 25 m, preferably 2 to 20 m, most preferably 5 to 20 m, such as 5 to 15 m.

In an embodiment, the heat exchangers each comprise 50 to 5000 tubes with an inner diameter in the range of 6 to 25 mm and an overall tube length of 2 to 30 m.

In an embodiment, the length of the heat exchangers is in the range of 10 to 30 m and the diameter of the shell is in the range of 0.5 to 2 m.

The stream of the hot chemical reaction product contains sensible heat from the chemical reaction. The sensible heat is potentially reclaimable energy that can be reused. In an embodiment, the withdrawn stream of the hot chemical reaction product has a temperature in the range of from 50 to 800° C., preferably 50 to 400° C., and an absolute pressure in the range of from 1 to 400 bar, preferably 20 to 400 bar, more preferably 50 to 400 bar.

In a preferred embodiment, the inventive method is a method for heat recovery during the production of isoprenol, more particularly for prolonging operation intervals between maintenance disruptions in such a method. In this process, a first reactant stream is an isobutylene stream and a second reactant is a stream of a formaldehyde source, and the chemical reaction product is an isoprenol-containing product. The isoprenol-containing product is heat-exchanged with the isobutylene stream.

Preferably, production of isoprenol is conducted under supercritical conditions. Supercritical conditions exist when a substance or mixture of substances is subjected to temperature and pressure exceeding the thermodynamic critical point of the substance or mixture. In this state, there is no differentiation between the liquid and gas phases and the fluid is referred to as a dense gas in which the saturated vapor and saturated liquid states are identical. It has been found that under supercritical conditions the reactivity of isobutylene towards formaldehyde is sufficiently high to allow for a smooth reaction even in the absence of an extraneous solvent or catalyst. This leads to a decreased amount of side products.

It is considered that supercritical isobutylene acts as a supercritical solvent which solubilizes at least part of the formaldehyde source and/or extracts formaldehyde out of the formaldehyde source. This allows for formaldehyde to be mixed with isobutylene efficiently, alleviating the problems generally associated with mixing multiple phases. For instance, local oversaturation of formaldehyde may be avoided. Thus, while ideally the entire reaction mixture, including the formaldehyde source and isobutylene, becomes a homogeneous single phase in the supercritical or near-supercritical region, it is also envisaged that part of the formaldehyde source exists as a second liquid phase. Also, the isoprenol formed in the reaction may dissolve in the near- or supercritical reaction mixture, or cause a separate liquid phase to form.

Forming a supercritical reaction mixture of isobutylene and formaldehyde requires supply of at least the isobutylene which is used in excess as both a reactant and solvent to the reactor at the necessary pressure and temperature. To achieve the necessary temperature of the isobutylene, energy consuming heaters are typically necessary. Increasingly more additional energy is required if pre-heating of the isobutylene by heat exchange with the hot isoprenol product is gradually reduced due to rapid and advanced fouling of the single heat exchanger typically used.

The phase diagrams of the reaction components are readily available to those skilled in the art. Further, the critical point of a compound or a mixture may be determined by calculating or experimentally obtaining a phase diagram of the compound or mixture. From the above, it is evident that the pressure and temperature of the process are required to be sufficient to maintain at least the isobutylene in a supercritical state. The critical temperature Tc and pressure Pc of isobutylene are Tc=417.9 K and Pc=40.00 bar (Tsonopoulos, C.; Ambrose, D., Vapor-Liquid Critical Properties of Elements and Compounds. 6. Unsaturated Aliphatic Hydrocarbons, J. Chem. Eng. Data, 1996, 41, 645-656).

In order to achieve supercritical conditions, formaldehyde and isobutylene are preferably reacted at a temperature of at least 220° C., for example in the range of 220 to 290° C., and an absolute pressure of at least 200 bar, more preferably at a temperature of at least 250° C., for example in the range of 250 to 280° C., and an absolute pressure of at least 220 bar, and most preferably at a temperature of at least 260° C., for example in the range of 260 to 275° C., and an absolute pressure of at least 250 bar. All pressures cited herein are absolute pressures, unless noted otherwise.

Preferably, a molar excess of isobutylene is reacted with formaldehyde. The term “molar excess of isobutylene” is understood to mean a molar excess of isobutylene over formaldehyde. This leads to a relatively low concentration of formaldehyde in the reaction mixture, thereby reducing the potential of a local oversaturation of formaldehyde. Thus, the amount of obtained side products is lowered. Further, it was found that the larger the molar ratio of isobutylene to formaldehyde, the lower the tendency of the reaction mixture to form multiple phases.

In a preferred embodiment, the molar ratio of isobutylene to formaldehyde under supercritical conditions is at least 7:1, especially preferred at least 10:1 and most preferably at least 12:1, for example at least 12.5:1.

Formaldehyde may be provided as a liquid, for example as a solution of paraformaldehyde. Preferably, the formaldehyde source is an aqueous formaldehyde solution. When formaldehyde is provided as an aqueous solution, the solution preferably comprises at least 15 wt.-%, more preferably at least 25 wt.-% and most preferably at least 35 wt.-%, for example at least 45 wt.-% or at least 50 wt.-%, of formaldehyde, based on the total weight of the aqueous solution of formaldehyde.

Adequate fluid mixing is important in the production of isoprenol. The production of isoprenol thus preferably involves mixing and injecting the formaldehyde source and isobutylene into a reactor through a plurality of nozzles operated in parallel. Preferably, the plurality of nozzles is arranged in the lid of a reactor. In order to allow for a high degree of mixing and to avoid dead zones, the nozzles should be flush with the lid.

Upon injection and mixing with the reactor contents, the formaldehyde source and isobutylene form a homogeneous single phase in the supercritical region. Isobutylene is either a supercritical fluid prior to its injection into the reactor, or is a liquefied or gaseous phase near its supercritical state before the injection into the reactor. Either way, heating of the isobutylene is required before it is injected, typically by a combination of pre-heating by heat exchange with the hot isoprenol product followed by an energy consuming heater directly before injection, with increasing energy consumption if the pre-heating by heat exchange is hampered by reduced heat transfer due to fouling of the heat exchanger.

Further details regarding the design of the reactor and reaction conditions may be found, e.g., in WO 2020/049111 A1.

The invention moreover relates to a plant for the production of a chemical reaction product, comprising:
a reactor having at least one reactant inlet for receiving at least one reactant stream and a reaction product outlet for withdrawing a stream of a hot chemical reaction product from the reactor;
at least two shell-and-tube heat exchangers, each of the heat exchangers comprising a plurality of tubes and a shell-side heat exchange passage; wherein the heat exchangers are connected in series with regard to both the shell-side flow and the tube-side flow; and are interconnected such that the hot chemical reaction product is directed through the tubes of the heat exchangers; and the reactant is guided through the shell-side passage;
optionally, a heater for further increasing the temperature of the reactant stream.

The plant may have prolonged operation intervals between maintenance disruptions and/or decreased energy consumption in case the heater is used; compared to an identical plant except for having a single shell-and-tube heat exchangers in place of at least two shell-and-tube heat exchangers in series.

In an embodiment, the plant is adapted to enable countercurrent flow of the hot reaction product from the reactor and the reactant through the heat exchangers.

One aspect of the inventions relates to a plant for the production of a chemical reaction product with prolonged operation intervals, wherein said plant includes a step of heat exchanging the stream of hot chemical reaction product and at least one reactant stream and preferably a subsequent heater of the at least one reactant stream before introducing the one or more reactant streams into the reactor and wherein the plant has at least two shell-and-tube heat exchangers in series for heat-exchanging the stream of hot chemical reaction product with at least one of the reactant streams and wherein the operation intervals are prolonged compared to a plant identical except for having a single shell-and-tube heat exchanger in place of at least two shell-and-tube heat exchangers in series of the inventive plant.

One further aspect of the inventions relates to a plant for the production of a chemical reaction product with decreased energy consumption, wherein said plant includes a step of heat exchanging the stream of hot chemical reaction product and at least one reactant stream and preferably a subsequent heater of the at least one reactant stream before introducing the one or more reactant streams into the reactor and wherein the plant has at least two shell-and-tube heat exchangers in series for heat-exchanging the stream of hot chemical reaction product with at least one of the reactant streams and wherein the energy consumption is decreased compared to a plant identical except for having a single shell-and-tube heat exchanger in place of at least two shell-and-tube heat exchangers in series of the inventive plant.

Where applicable, the above embodiments regarding the process of the invention also valid for the plant of the invention.

A further embodiment is the use of at least two shell-and-tube heat exchangers in series in a plant for production of a chemical reaction product including a step of heat exchanging the stream of hot chemical reaction product and another stream, for example a reactant stream to increase the intervals between maintenance disruptions compared to an identical plant except it has a single shell-and-tube heat exchanger in place of at least two shell-and-tube heat exchangers in series.

Yet a further embodiment of the invention is the use of at least two shell-and-tube heat exchangers in series in a plant for production of a chemical reaction product including a step of heat exchanging the stream of hot chemical reaction product and at least one reactant stream and a subsequent heater of the at least one reactant stream to decrease the energy consumption compared to an identical plant except it has a single shell-and-tube heat exchanger in place of at least two shell-and-tube heat exchangers in series.

The invention is further illustrated by the examples and figures that follow.

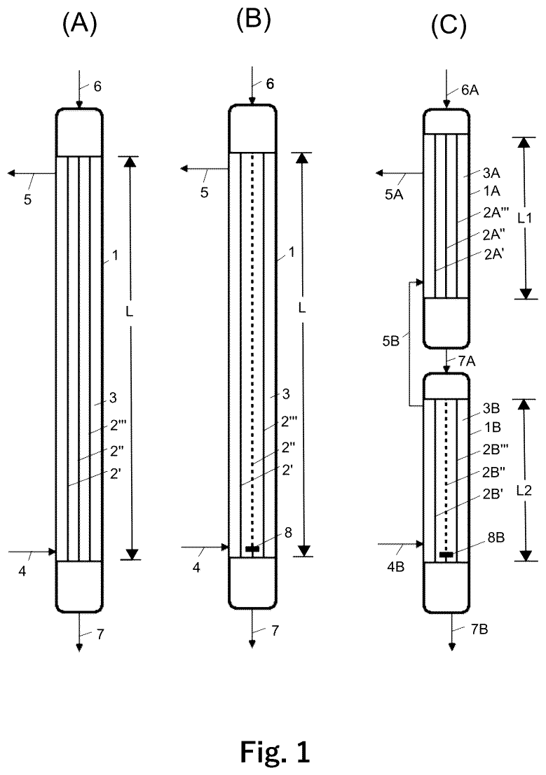

FIG. 1(A) to (C) depict schematic illustrations of different operational states and arrangements of shell-and-tube heat exchangers.

FIG. 1(A) depicts a schematic illustration of a shell-and-tube heat exchanger 1 comprising a cylindrical shell and a plurality of tubes 2′, 2″, 2′″ arranged within the shell. A shell-side heat exchange passage 3 allows for circulating a heat transfer liquid in the space between the tubes and within the shell. The heat exchange passage 3 may comprise baffles (not shown) to optimize flow of the heat transfer liquid through the heat exchange passage 3.

A stream of a hot chemical reaction product, e.g. obtained from a chemical reaction in a reactor (not shown), is introduced via line 6 and distributed via an upper tube sheet to the tubes 2′, 2″, 2′″ of the shell-and-tube heat exchanger 1. The tubes 2′, 2″, 2′″ have an overall length L from the upper tube sheet to the lower tube sheet. Simultaneously, a reactant is introduced via line 4 into and guided through the shell-side passage 3 of the shell-and-tube heat exchanger 1. Heat is transferred from the hot chemical reaction product to the reactant stream through the walls of the tubes 2′, 2″, 2′″.

The stream of the chemical reaction product is withdrawn through line 7 of the shell-and-tube heat exchanger 1 at the bottom part of the shell-and-tube heat exchanger 1, wherein said stream withdrawn through line 7 has a lower temperature than the stream of the hot chemical reaction product introduced via line 6. The pre-heated reactant is withdrawn through line 5 of the shell-and-tube heat exchanger 1 at the top part of the shell-and-tube heat exchanger 1 and can be directed to the reactor (not shown) or further heated if required. The reactant withdrawn through line 5 has a higher temperature than the reactant introduced via line 4.

FIG. 1(B) depicts the same shell-and-tube heat exchanger 1 as shown in FIG. 1(A) and illustrates an operational situation wherein the tube 2″ is clogged near to the lower tube sheet by a blockage 8. This results in a reduced flow of the stream of the hot chemical reaction product through the tube 2″ or liquid stagnation in the tube 2″ above the lower tube sheet. Thus, essentially the entire wall surface of the tube 2″ is no longer available for heat transfer and the heat transfer efficacy of the shell-and-tube heat exchanger 1 is reduced.

FIG. 1(C) depicts an arrangement of two shell-and-tube heat exchangers in series comprising a first shell-and-tube heat exchanger 1B and a second shell-and-tube heat exchanger 1A.

Seen in the flow direction of the at least one reactant, the first shell-and-tube heat exchanger 1B comprises a plurality of tubes 2B′, 2B″, 2B′″ and a shell-side heat exchange passage 3B. The tubes 2B′, 2B″, 2B′″ of the first shell-and-tube heat exchanger 1B have an overall length L2 from the upper tube sheet to the lower tube sheet.

The second shell-and-tube heat exchanger 1A in the flow direction of the one or more reactants comprises a plurality of tubes 2A′, 2A″, 2A′″ and a shell-side heat exchange passage 3A. The tubes 2A′, 2A″, 2A′″ of the second shell-and-tube heat exchanger 1A have an overall length L1 from the upper tube sheet to the lower tube sheet.

Each of the lengths L1 and L2 is less than the length L. In the illustrated embodiment, the sum of L1 and L2 approximately equals L.

The first shell-and-tube heat exchanger 1B and the second shell-and-tube heat exchanger 1A are connected in series with regard to both the shell-side flow and the tube-side flow. The hot reaction product is introduced into the second shell-and-tube heat exchanger 1A via line 6A and distributed via an upper tube sheet to tubes 2A′, 2A″, 2A′″. The partially cooled down product emerging from the tubes 2A′, 2A″, 2A′″ of the second shell-and-tube heat exchanger 1A is introduced via line 7A into the first shell-and-tube heat exchanger 1B and distributed via an upper tube sheet to the tubes 2B′, 2B″, 2B′″. Conversely, the reactant to be pre-heated is introduced via line 4B into the shell-side passage 3B of the first shell-and-tube heat exchanger 1B and after leaving the same is directed into the shell-side passage 3A of the second shell-and-tube heat exchanger 1A via line 5B. The pre-heated reactant is withdrawn through line 5A.

During operation, fouling may occur, causing a blockage 8B in the tube 2B″. This results in the tube 2B″ being partially blocked or fully blocked, i.e. in a reduced flow of the stream of a hot chemical reaction product through the tube 2B″. Only the wall surface of tube 2B″ is no longer available for heat transfer. Thus, in comparison to FIG. 1(B), a larger surface of the tube wall is still available for heat transfer even in case of a blockage of an individual tube.

It is also possible in some chemical production processes that the fouling may occur by partial or fully blocking of a tube in the heat exchanger 1A, for example 2A″. That would analogously be 8A if it would be included in the FIGURE. In such a case, the invention allows the still partially hot reaction product to make use of the full wall surface of the heat exchanger 1B including the tube 2B″, while in the case of a single shell-and-tube heat exchanger as shown in FIG. 1(A), fouling in the upper half of a tube like tube 2″ will effectively result in the loss of the entire wall surface of tube 2″ over the full length of the heat exchanger with respect to heat transfer to the at least one reactant stream in the shell surrounding tube 2″.

It has to be understood that the inventive arrangement of at least two shell-and-tube heat exchangers in series as shown in FIG. 1 is not limiting the invention to physically separated at least two such heat exchangers in series. For example, these might be within one casing. Also, in one aspect of the invention said at least two heat exchangers in series is to be understood to comprise a set-up that resembles a single heat exchanger at first glance, except that it has in the middle portion a zone where the partially cooled reaction product streams running through the tubes of one part of a heat exchange unit are reunited and then again split into streams fed through further tubes in the other part of the shell-and-tube heat exchange unit for further transfer of heat to the at least one reactant entering that part of the heat exchange unit in need of pre-heating—by this creating two shell-and-tube heat exchangers in a single heat exchange unit.

### EXAMPLE

For the industrially important conversion of isobutylene and formaldehyde yielding isoprenol, isobutylene must be pre-heated. For this purpose, the hot product isoprenol is heat exchanged with isobutylene.

The heat exchange performance between a hot isoprenol stream and a cold isobutylene stream was calculated by applying correlations of heat transfer which are documented in VDI-Wärmeatlas, VDI-Gesellschaft Verfahrenstechnik und Chemieingenieurwesen Hrsg., 11., bearbeitete und erweiterte Auflage (e.g. chapter G1 “Durchströmte Rohre”; Volker Gnielinski, Institut für thermische Verfahrenstechnik, Karlsruher Institut far Technologie”, page 785-791).

Heat exchange was simulated according to the following scenarios:
(1) single shell-and-tube heat exchanger (reference example) having 1942 tubes with an outer diameter of 14 mm, an inner diameter of 8 mm and a length of 14 m, resulting in a heat exchange surface of 1025 m²,
(2) two shell-and-tube heat exchangers connected in series (example in accordance with the invention) with a total heat exchange surface area of 1025 m²:
   first shell-and-tube heat exchanger having 1942 tubes with an outer diameter of 14 mm, an inner diameter of 8 mm and a length of 7 m, resulting in a heat exchange surface of 512.5 m², and
   second shell-and-tube heat exchanger having 1942 tubes with an outer diameter of 14 mm, an inner diameter of 8 mm and a length of 7 m, resulting in a heat exchange surface of 512.5 m².

The following specifications apply:
isobutylene: mass flow=81.1 t/h, and
isoprenol: mass flow=87.9 t/h.

The results of the simulation are shown in Table 1. “Blockage” relates to the percentage of tubes failing due to fouling. These tubes were deemed to contribute no longer to the heat exchange performance.

TABLE 1

| | | | isobutylene (“reactant”) | | isoprenol (“product”) | | | | |
|---|---|---|---|---|---|---|---|---|---|
| | | blockage | T inlet | T outlet | T inlet | T outlet | heat exchange | Δ h.e. ^{[1]} | k-value |
| # | Scenario | [%] | [° C.] | [° C.] | [° C.] | [° C.] | [kW] | [%] | [W/(m² · K)] |
| 1 | (1) | 0 | 60.0 | 276.2 | 285.0 | 122.4 | 13391 | 0 | 477 |
| 2 | (1) | 60 | 60.0 | 264.2 | 285.0 | 133.5 | 12567 | −6.2 | 293 |
| 3 | (1) | 80 | 60.0 | 246.9 | 285.0 | 148.8 | 11391 | −14.9 | 185 |
| 4 | (2) | 0 (1^{st} h.e. ^{[2]}) | 60.0 | 219.7 | 238.6 | 122.4 | 9577 | 0 | 513 |
| | | 0 (2^{nd} h.e. ^{[3]}) | 219.7 | 276.2 | 285.0 | 238.6 | 3815 | | 563 |
| | | (total) | — | — | — | — | 13392 | | 477 |
| 5 | (2) | 60 (1^{st} h.e. ^{[2]}) | 60.0 | 184.9 | 214.6 | 126.5 | 7326 | −2.2 | 313 |
| | | 0 (2^{nd} h.e. ^{[3]}) | 184.9 | 271.8 | 285.0 | 214.6 | 5768 | | 554 |
| | | (total) | — | — | — | — | 13094 | | 388 |
| 6 | (2) | 0 (1^{st} h.e. ^{[2]}) | 60.0 | 230.1 | 250.7 | 126.6 | 10269 | −2.3 | 511 |
| | | 60 (2^{nd} h.e. ^{[3]}) | 230.1 | 271.7 | 285.0 | 250.7 | 2819 | | 330 |
| | | (total) | — | — | — | — | 13088 | | 386 |
| 7 | (2) | 60 (1^{st} h.e. ^{[2]}) | 60.0 | 197.0 | 230.5 | 133.6 | 8097 | −6.1 | 310 |
| | | 60 (2^{nd} h.e. ^{[3]}) | 197.0 | 264.2 | 285.0 | 230.5 | 4473 | | 327 |
| | | (total) | — | — | — | — | 12570 | | 293 |
| 8 | (2) | 80 (1^{st} h.e. ^{[2]}) | 60.0 | 154.2 | 193.8 | 129.8 | 5409 | −4.1 | 198 |
| | | 0 (2^{nd} h.e. ^{[3]}) | 154.2 | 268.2 | 285.0 | 193.8 | 7438 | | 546 |
| | | (total) | — | — | — | — | 12847 | | 337 |
| 9 | (2) | 95 (1^{st} h.e. ^{[2]}) | 60.0 | 99.2 | 161.9 | 134.9 | 2163 | −6.8 | 62 |
| | | 0 (2^{nd} h.e. ^{[3]}) | 99.2 | 262.8 | 285.0 | 161.9 | 10311 | | 516 |
| | | (total) | — | — | — | — | 12474 | | 281 |

^{[1]} Δ h.e. (heat exchange): reduction of heat exchange [kW] compared to 0% blockage.
^{[2]} first heat exchanger
^{[3]} second heat exchanger

1.-19. (canceled)

20. A method for prolonging operation intervals between maintenance disruptions in the production of a chemical reaction product, comprising
directing at least one reactant stream into a reactor;
reacting the at least one reactant in the reactor at elevated temperature and pressure, whereby the chemical reaction product is obtained;
withdrawing a stream of a hot chemical reaction product from the reactor; and
heat-exchanging the stream of hot chemical reaction product with at least one of the reactant streams;
wherein heat-exchanging is performed in at least two shell-and-tube heat exchangers; each of the heat exchangers comprising a plurality of tubes and a shell-side heat exchange passage;
wherein the hot chemical reaction product is directed through the tubes of the heat exchangers; and the reactant is guided through the shell-side passage, and at least two of the heat exchangers are connected in series with regard to both the shell-side flow and the tube-side flow.

21. The method according to claim 20, wherein the hot chemical reaction product is prone to fouling during heat exchange.

22. The method according to claim 20, wherein the heat transfer of the first heat exchanger, relative to the direction of the at least one reactant stream, gradually deteriorates.

23. The method according to claim 20, wherein the reduction of the overall heat transfer coefficient k of all heat exchangers connected in series is less than the reduction of the heat transfer coefficient k of the first heat exchanger, relative to the direction of the at least one reactant stream, preferably by at least 10 percentage points.

24. The method according to claim 20, wherein the hot chemical reaction product undergoes an at least partial phase change during the heat exchange.

25. The method according to claim 20, wherein heat exchanging reduces the temperature of the stream of hot chemical reaction product by at least 100° C.

26. The method according to claim 20, wherein the ratio of the heat exchange surface area of each individual heat exchanger relative to the heat exchange surface area of each of the other individual heat exchangers is in the range of 1:10 to 10:1.

27. The method according to claim 20, wherein heat-exchanging is performed in 2 to 4 heat exchangers connected in series.

28. The method according to claim 20, wherein the heat exchangers each comprise 50 to 5000 tubes with an inner diameter in the range of 6 to 25 mm and an overall tube length of 2 to 30 m.

29. The method according to claim 20, wherein the length of the heat exchangers is in the range of 10 to 30 m and the diameter of the shell is in the range of 0.5 to 2 m.

30. The method according to claim 20, wherein the stream of the hot chemical reaction product has a temperature in the range of from 50 to 800° C. and an absolute pressure in the range of from 1 to 400 bar.

31. The method according to claim 20, which is a method for heat recovery during the production of isoprenol, wherein a first reactant stream is an isobutylene stream and a second reactant is a stream of a formaldehyde source, wherein the chemical reaction product is an isoprenol-containing product, and the isoprenol-containing product is heat-exchanged with the isobutylene stream.

32. The method according to claim 31, comprising reacting the formaldehyde source and isobutylene at a temperature of at least 220° C. and an absolute pressure of at least 200 bar.

33. The method according to claim 31, comprising reacting a molar excess of isobutylene with the formaldehyde source, calculated as formaldehyde.

34. The method according to claim 31, wherein the formaldehyde source is an aqueous formaldehyde solution.

35. The method according to claim 34, wherein the aqueous formaldehyde solution comprises at least 15 wt.-%, of formaldehyde, based on the total weight of the aqueous solution of formaldehyde.

36. The method according to claim 31, wherein the formaldehyde source and isobutylene are reacted in the essential absence of a catalyst.

37. A plant for the production of a chemical reaction product, comprising:
a reactor having at least one reactant inlet for receiving at least one reactant stream and a reaction product outlet for withdrawing a stream of a hot chemical reaction product from the reactor;
at least two shell-and-tube heat exchangers, each of the heat exchangers comprising a plurality of tubes and a shell-side heat exchange passage; wherein the heat exchangers are connected in series with regard to both the shell-side flow and the tube-side flow; and are interconnected such that the hot chemical reaction product is directed through the tubes of the heat exchangers; and the reactant is guided through the shell-side passage;
optionally, a heater for further increasing the temperature of the reactant stream;
wherein the plant has prolonged operation intervals between maintenance disruptions and/or decreased energy consumption in case the heater is used; compared to an identical plant except for having a single shell-and-tube heat exchangers in place of at least two shell-and-tube heat exchangers in series.

38. The plant according to claim 37, wherein the hot reaction product from the reactor and the reactant are flowing countercurrently through the heat exchangers.
